(19) United States
(12) Patent Application Publication
(10) Pub. No.: US 2018/0055835 A1
(43) Pub. Date: Mar. 1, 2018

(54) Method for Treating And Preventing Protozoal Infections

(71) Applicant: Immune Therapeutics Inc., Orlando, FL (US)

(72) Inventors: Noreen Griffin, Winter Park, FL (US); Fengping Shan, Shenyang (CN)

(21) Appl. No.: 15/685,706

(22) Filed: Aug. 24, 2017

Related U.S. Application Data
(60) Provisional application No. 62/379,272, filed on Aug. 25, 2016.

(51) Int. Cl. A61K 31/485; A61K 45/06

(52) U.S. Cl. CPC A61K 31/485; A61K 45/06

(57) ABSTRACT

A method for treating or preventing protozoan parasite infection comprising administering to a mammal in need thereof naltrexone or a pharmaceutically acceptable salt thereof alone or in combination with one or more anti-protozoal agents is provided.

### CROSS-REFERENCE TO RELATED APPLICATIONS

This application claims priority under 35 U.S.C. §119(e) to Provisional U.S. Patent Application Ser. No. 62/379,272, filed Aug. 25, 2016. The disclosure of the aforementioned application is incorporated by reference in its entirety.

### FIELD OF THE INVENTION

The present invention relates to methods of treating and preventing protozoal infections comprising administering naltrexone, preferably as a single oral tablet, capsule, liquid or cream dose of naltrexone, either alone or in combination with one or more anti protozoal agents.

### BACKGROUND

Protozoal parasites are single-celled organisms which live during some or all stages of their life cycle within organs, tissues and cells of mammals. As parasites, they obtain nutrients either from the host organism's food supply or from its cells and tissues. As eukaryotic unicellular organisms, the protozoal parasites are able to live both within animal cells and as free living extra-cellular parasites residing in the blood, lymph tissue or within the intestinal lumen.

As agents of infection, the protozoal parasites are fundamentally different than bacteria and viruses. Unlike bacteria and viruses, protozoa parasites are animals and share similar metabolism, respiration, and nutritional needs with their animal hosts. The similar metabolism of protozoa to mammalian metabolism renders most antibiotics and antiviral agents, selectively active against bacteria and viruses, respectively, ineffective for protozoal infection. Activity of compounds with antibacterial or antiviral activity against protozoal parasites would be atypical and unexpected. The lack of differences between protozoal metabolism and host cellular metabolism requires novel pharmacologic approaches to find therapeutic agents selective for eliminating protozoal organisms living within an animal host. Unlike bacteria and viruses, protozoa may assume different sexual forms and differentiate into a variety of maturational stages in various organs, presenting unique challenges for recognition by the host immune system. As genetically more complex organisms than bacteria and viruses, protozoa differentiate into forms which resist killing by known microbicides active against bacteria and viruses (Weir et al., 2002, Appl Environ Microbiol. 68(5):2576-9).

The primary protozoal parasites causing disease in man include hemoflagellates of the class Trypanosomatidea, causing Leishmaniasis and Trypanosomiasis, and parasites of the phylum Apicomplexa, class Coccidea, causing malaria, toxoplasmosis, cryptosporidiosis, and bebesiosis. Species of Coccidea can infect humans, domestic animals and livestock, including poultry, lambs, calves, piglets, and rabbits. Protozoal parasitic diseases related to malaria include disease caused by parasites of the species Neospora. Neospora infections occur in dogs, cattle, sheep, goats and horses. The majority of populations in developing countries are now at high risk of various protozoal infections including malaria, leishmaniasis, and trypanosomiasis. Together these protozoal diseases cause millions of preventable deaths every year. No preventive or therapeutic vaccines are yet available for these parasitic diseases. The market for drugs against such diseases is limited by poverty and the emergence of resistance to existing single agent chemotherapy. As used herein, chemotherapy refers to the use of chemical substances to treat disease. Due to the lack of protective immunity following infection, with or without chemotherapy, reinfection is a common phenomenon. Innovative and cost effective new drugs and combination therapies using new and old drug products are urgently needed. The development of broad-spectrum anti-parasitic agents able to be used in combination with existing chemotherapeutics is preferable to reduce the emergence of new resistance. An ideal anti-protozoal drug would target multiple protozoan parasites, be active by various routes of administration, reduce morbidity and mortality caused by such infections, not interfere with co-administered vaccines as they become available, and reduce the need for hospital-based treatment.

Parasitic protozoa are responsible for a variety of human diseases transmitted by insect vectors, i.e., carriers, including malaria, leishmaniasis, and trypanosomiasis. Other protozoal parasites can be transmitted directly from other mammalian reservoirs or from person to person. Lacking vaccines, vector control and selective chemotherapy have been the only ways to reduce transmission and treat infected individuals, respectively. Because the immune system plays a crucial role in controlling protozoal infection, opportunistic infection with protozoal organisms is an increasing problem in infants, cancer patients, transplant recipients, and those co-infected with human immunodeficiency virus (HIV). Pregnancy also suppresses certain immune functions. New anti-protozal treatments are needed which are safer for mother and fetus during pregnancy, particularly for malaria, toxoplasmosis, and trichomonas infections. Vaccines are needed which overcome diminished immune responses and induce an adequate long term immune response. Vaccines can be used in conjunction with compatible chemotherapy to improve therapy of pre-existing chronic infection in endemic areas.

Malaria arises from infection with an Apicomplexan protozoan parasite known as Plasmodium. Only four species of the genus Plasmodium cause human malaria. P. vivax is the most common and fatal. P. ovale and P. malariae are less common and have intermediate severity. P. falciparum is the most virulent, responsible for high infant mortality, and associated with current drug resistance. The disease is transmitted to human beings through the bite of infected female Anopheles mosquitoes and by transfusion of infected blood.

Due to the emergence and spread of drug-resistant malaria parasites, pesticide-resistant malaria-transmitting mosquitoes, and population growth in endemic areas, malaria now causes approximately 500 million clinical cases per year. It is prevalent in children and pregnant women, causing about one million annual deaths in children under the age of five. Children growing up in rural and endemic areas are subject to more frequent malaria related illness and deaths than more resistant adults.

The most severe form of Plasmodium falciparum infection is cerebral malaria (CM). Cerebral malaria implies the presence of neurological features, especially impaired consciousness. Treatment of CM is limited to a few conventional anti-malarial drugs (quinine or artemisinins) and supportive care including parenteral fluids, blood exchange transfusion, osmotic diuretics and correction of hypoglycemia, acidosis and hypovolemia. The management of CM includes prompt administration of appropriate parenteral anti-malarial agents and early recognition and treatment of the complications. In children, the complications include severe anemia, seizures and raised intracranial pressure. In adults, renal failure and pulmonary edema are more common causes of death.

A number of drugs ranging from those of natural origin to synthetic ones have been developed for the treatment of malaria. Quinine and artemisinin are the commonly known drugs of natural origin, which are used for the treatment of malaria. A number of synthetic anti-malarial drugs such as chloroquine, mefloquine, primaquine, halofantrin, amodiaquine, proguanil, atovaquone, maloprim are known in the literature. Quinidine Gluconate, Quinine Sulfate, typically in combination with Doxycycline hyclate, Clindamycin, or Pyrimethamine-sufadoxine are also used for malaria. In chloroqine resistant strains, preferred oral therapy includes Mefloquine Hydrochloride and Atovaquone-proguanil hydrochloride combinations. In treatment of infections with P. vivax, P. malariae, P. ovale, and chloroquine sensitive P. falciparum, chloroquine phosphate and primaquine phosphate are used.

In recent years, drug resistant malaria has become one of the most serious problems in malaria control. Drug resistance necessitates the use of drugs which are more expensive and may have dangerous side effects. The emergence of resistance can be prevented by the use of combinations of drugs with different mechanisms of action. The use of drug combinations for all antimalarial treatment not only delays the onset of drug resistance, but also accelerates recovery and increases cure rates. A number of antimalarial combinations are already known in the field of malarial chemotherapy. The specific combinations in use, dosages, and relative merits of various combinations have been summarized (Kremsner et al., 2004, Lancet 364:285-94).

With the emergence of P. falciparum strains resistant to chloroquine and quinine, further alternative antimalarial chemotherapy is required. Due to frequent re-infection following complete or partial treatment, vaccine therapy promoting long term immunity to re-infection is needed. New chemotherapy will preferably clear the current infection and not interfere with co-administered vaccines as they become available. Preferred combinations of anti-malarials utilize drugs that overcome chloroquine resistance, have a good safety profile, and are well tolerated. Artemisinin, obtained from the plant Artemisia anua, and its derivatives are rapidly effective in severe malaria. Artemisinin compounds have been evaluated in several centers and are found to be effective, and safe (Miskra et al., 1995, Trans R Soc Trop Med Hyg 89:299-301).

In addition, the patent literature describes the combination of atovaquone and proguanil as a method for the treatment of malaria. See U.S. Pat. No. 5,998,449. The combination of fenozan with another anti-malarial agent selected from artemisinin, sodium artesunate, chloroquine, or mefloquine is described for the prophylactic and curative treatment of malaria. See U.S. Pat. No. 5,834,505. Synergistic combination kits using atemisinin derivatives, sulfadoxin and pyrimethamine for severe, multi-drug resistant malaria are described by Tipathi et al. in U.S. Patent Application Publication No. 2006/0141024 A 1.

African trypanosomiasis (sleeping sickness) is caused by a subspecies of the parasitic haemoflagellate, Trypanosoma brucei. The infection begins with the bite of an infected tsetse fly (Glossina spp.). Two forms of the disease are known, one caused by Trypanosoma brucei rhodesiense, endemic in Eastern and Southern Africa, and the other caused by T. b. gambiense, originally detected in West Africa, but also widespread in Central Africa. African Trypanosomiasis results in febrile, life-threatening illness in humans and also threatens livestock. T. brucei parasites rapidly invade the Central Nervous System (CNS) causing death within weeks if untreated. T. b. gambiense proliferates relatively slowly and can take several years before infecting the CNS system. There are four important drugs approved to treat these infections. Two of these, pentamidine and suramin, are used before the CNS involvement. The arsenic-based drug, melarsoprol is used in the case of infections established in the CNS. The fourth drug, eflornithine, is used against late stage infection caused by T. b. gambiense. This drug is ineffective against T. b. rhodesiense. Nifurtimox is another drug licensed for both American trypanosomiasis and melarsoprol-refractory late stage disease.

American trypanosomiasis or Chaga's disease is caused by Trypanosoma cruzi and effects millions of people in South and Central America, and Mexico. Untreated Chaga's disease causes decreased life expectancy due to parasitic cardiomyopathy and heart failure, megaesophagus, and megacolon. Blood-sucking triatomid bugs transmit the infection to young children and transplacental infection can occur with parasitemia during pregnancy. Nifurtimox and benznidazole are two drugs used for treatment of the acute disease, but are not known to be therapeutic for the chronic infection in older children and adults. In the absence of an effective vaccine, better agents are needed that can be taken prophylactically by at risk children. Following infection, additional agents are needed to be used in conjunction with nifurtimox and benznidazole to increase efficacy, permit lower doses of the current agents with reduced toxicity, and shorten the currently required duration of treatment.

Human leishmaniasis comprises a heterogeneous spectrum of diseases. Three major forms are generally distinguished: cutaneous leishmaniasis, mucocutaneous leishmaniasis and visceral leishmaniasis, of which the latter is potentially lethal. They are caused by various species of the protozoan parasite Leishmania and transmitted by female sandflies. The disease is currently estimated to affect some 12 million people in 88 countries. Worldwide, leishmania/HIV co-infection is now considered an emerging disease where about 50% of adult visceral leishmaniasis cases are related to co-existing HIV infection.

The current treatment for leishmaniasis involves administration of pentavalent antimony complexed to a carbohydrate in the form of sodium stibogluconate (Pentosam or Sb(V)) or meglumine antimony (Glucantine), which are the only established anti-leishmanial chemotherapeutic agents with a clearly favorable therapeutic index. The exact chemical structure and mode of action of pentavalent antimonials is still uncertain. Amphotericin B and Pentamidine are the second line of anti-leishmanial agents, but are reserved for non-responding infections due to potential toxicity. Since resistance to the antimony-based anti-Leishmanial drugs is emerging and treatment failures are common, new combination therapies are needed. Miltefosine is a recently introduced oral drug effective for visceral and cutaneous disease. The importance of this new oral agent extends to the treatment of dogs which serve as an important reservoir of the disease. The identification of additional, new and effective anti-leishmanial agents for oral administration would allow further treatment options, help prevent emerging resistance to Miltefosine and antimony-based drugs, and increase the chance for regional control of leishmaniasis. DIM has been shown to be a potent inhibitor of Leishmania donovani topoisomerase I (LdTOPILS) with an IC50 of 1.2 micromolar. See Roy A., et al., Biochemical Journal, 8 Oct. 2007, Immediate Publication Manuscript BJ 20071286 (not the final version).

Trichomonal infection, typically vulvo-vaginitis in women and urethritis in men, is sexually acquired and one of the most common protozoal parasite infections in humans. In the United States, it is estimated that more than 2 million women are infected each year. Trichomonas vaginitis causes vulvar itching and an odorous vaginal discharge. It is caused by Trichomonas vaginalis, a single-celled protozoan parasite not normally found in the flora of the genitourinary tract. Typically, Trichomonal infection is treated with oral metronidazole which is FDA approved in various dosage regimens. Though efficacious, Metronidazole can exhibit serious dose-related side effects, particularly on the blood and on the central nervous system. Experiments show it to be mutagenic and carcinogenic. Recently, treatment failure and emerging resistance to metronidizole have been documented, indicating a need for more consistently effective therapies which will include combinations of drugs active against strains of T. vaginalis that may be resistant to metronidazole. Preferred treatments will include agents safe for pregnant women and allow lower doses of co-administered metronidazole.

The risk of parasitic diseases is also present outside developing countries and often takes the form of chronic diarrheal disease in subjects with underlying immune deficiency. These infections can be caused by Isospora belli, and Cyclospora cayetanensis, both coccidian protozoa, where infection results in self-limited diarrhea in normal hosts and prolonged diarrhea in individuals with AIDS. Both infections respond to treatment with timethroprim-sufamethoxazole. Cryptosporidia are additional coccidian parasites that cause diarrhea in animal species and humans. Cryptosporidium parvum and C. Hominis account for most coccidial infections in humans. These organisms form oocytes, which when digested release sporozoites that invade host epithelial cells, penetrating the cell membrane but not the enterocyte cytoplasm. Nitazoxanide is the only drug approved for the treatment of cryptosporidiosis in the United States. The identification of additional effective anti-crytosporidial agents for oral use would allow additional treatment options for individuals with HIV infection who respond unpredictably to Nitazoxanide.

Toxoplasmosis, is a zoonotic infection by the obligate intracellular protozoan, Toxoplasma gondii. Toxoplasmosis is found throughout the world, including the United States. Cats and other feline species are the natural hosts for Toxoplasma gondii, however tissue cysts (bradyzoites) have been recovered from all mammalian species examined Pregnant women and those with weak immune systems are particularly susceptible to the health risks resulting from Toxoplasma infection. Severe toxoplasmosis, particularly trans-placental exposure, can result in damage to the brain, eyes, and other developing organs in utero. Currently available treatments for toxoplasmosis, which are the drugs trisulfa-pyrimdine, sulfadiazine and pyrimethamine, are not effective, and can be toxic to the host. Therefore, there is a need for therapeutic agents to treat toxoplasmosis that are more effective and less toxic than currently available treatment agents. No available agent is used to control Toxoplasmosis in cats.

Accumulating evidence suggests that immediate release low dose naltrexone between 0.05 and 6 mg can promote health supporting immune modulation which may reduce various oncogenic and inflammatory autoimmune processes through a number of receptors including the opiate, TIM-3 Tolling and PD-1 as the mediated mechanism. By targeting the opiate and PD-1 pathways, T cell exhaustion is reversed and anti-tumor immunity is restored.

All of these receptors play a key roll in immune deregulation or immune dysfunction When an immediate release low dose of naltrexone between 0.05 and 6 mg has been shown to be useful in the treatment of HIV/AID's fibromyalgia, Crohn's disease, multiple sclerosis and localized pain syndromes. Chronic HIV infection is associated with persistent inflammation and expression of negative check point receptor (NCR) (eg: opiate, tolling, p16, pathway, PD-1 pathway TIM-3 and TIGIT) on T Cells that result in immune deregulation, immune dysfunction or T-Cell Exhaustion. The connection between patients with HIV and chronic malaria is very easy to understand as both are affected by T-cell exhaustion.

Immediate release low dose naltrexone between 0.05 mg and 6 mg can provide a new, safe and inexpensive method of medical treatment by mobilizing the natural defenses of one's own immune system through binding to a number of the bodies receptors including the opiate receptor, tolling receptor, p16 pathway, PD-1 pathway, TIM-3 and TIGIT, endothelial protein C receptor.

Malaria is a highly prevalent disease caused by infection by Plasmodium spp., which infect hepatocytes and erythrocytes and is a blood-stage infections cause devastating symptoms and can persist for years. Antibodies and CD4+ T cells are thought to protect against blood-stage infections. The components of the immune system responsible for killing the Plasmodium parasites remain unclear, although antibodies are known to have a key role in controlling blood-stage infections (Cohen et al., 1961).

Furthermore, studies in experimental rodent models showed that CD4+Th1-immune responses are also a critical component of protection against chronic blood-stage malaria (Kumar and Miller, 1990, Stephens and Langhorne, 2010, Su and Stevenson, 2002). Finally, depletion of CD8+ T cells delayed clearance of P. chabaudi malaria, implicating these cells in protection against chronic disease (Podoba and Stevenson, 1991).

In addition to the antagonist effect on opiate receptors, LDN simultaneously has an antagonist like effect on non-opioid receptors including. TRL4; TRL9, PD-1 and p16 Receptors. In order for the body to maintain good health and wellness, there is a balance of the immune system between the cellular (Th1) and the humoral (Th2) immune systems Immune imbalance is regulated through T-helper cells that produce cytokines. The Th1 lymphocytes help fight pathogens that are within cells like cancer and viruses through activation of interferon-gamma and macrophages. The Th2 lymphocytes target external pathogens like parasites, allergens, toxins through the activation of B-cells and antibody production. IRLDN has proven to assist with immune dysfunction, immune dysregulation and T-cell exhaustion. There is therefore a need for agents that treat or prevent protozoal infections. It has surprisingly found that naltrexone treats and prevents infection by protozoans.

### SUMMARY OF THE INVENTION

The present invention relates to a method for treating or preventing protozoan parasite infection comprising administering to a mammal or bird in need thereof naltrexone or a pharmaceutically acceptable salt thereof alone or in combination with one or more anti-protozoal agents such as atovaquone, amodiaquine, amphotericin, butoconazole, clindamycin, eflornithine, fumagillin, iodoquinol (diiodohydroxyquin), clioquinol (iodochlorhydroxyquin), Etanidazole, Benznidazole, fluoroquinolones, enoxacin, ciprofloxacin, doxycycline, tetracycline, melarsoprol, metronidazole, miltefosine, nifurtimox, nitazoxanide, paromomycin, pentamindine, sodium stibogluconate, suramin, tinidozole, pyrimethamine, proguanil (chloroguanide), spiramycin, sulfadoxine, artemisinin, dihydroartemisinin, artemether, artesunate, quinine derived from the bark of the South American chinchona tree, including quinine and quinine-related quinolines, halofantrine, mefloquine, lumefantrine, amodiaquine, pyronaridine, piperaquine, chloroquine, hydryoxychloroquine, napthoquine, primaquine, tafenoquine, 6-gingerol and 6-paradol, coronaridine and 18-methoxycoronaridine.

In certain embodiments the infection to be treated by the method is caused by a protozoan parasite of the genera Giardia, Trichomonas, Leishmania, Trypanosoma, Crithidia, Herpetomonas, Leptomonas, Histomonas, Eimeria, Isopora, Neospora, or Plasmodium.

In certain embodiments the infection to be treated is malaria.

The present invention provides a method for treating or preventing protozoan parasite infection, producing protective antibodies to malaria, restoring CD4+ T cell function, amplifying follicular helper T-cells and germinal center B cells or increasing CD3+ T cell, CD8+ T cell, CD4+ T cell, NK cell, dendritic cell and/or macrophages comprising administering to a mammal or bird, preferably a human, in need thereof an immediate release or sustained release pharmaceutical formulation, preferably comprising between about 0.01 mg and about 10.0 mg of naltrexone (low dose naltrexone, LDN or immediate release low dose naltrexone IRLDN) or a pharmaceutically acceptable salt thereof, preferably a hydrochloride salt, alone or in combination with one or more anti-protozoal agents such as atovaquone, amodiaquine, amphotericin, butoconazole, clindamycin, eflornithine, fumagillin, iodoquinol (diiodohydroxyquin), clioquinol (iodochlorhydroxyquin), Etanidazole, Benznidazole, fluoroquinolones, enoxacin, ciprofloxacin, doxycycline, tetracycline, melarsoprol, metronidazole, miltefosine, nifurtimox, nitazoxanide, paromomycin, pentamindine, sodium stibogluconate, suramin, tinidozole, pyrimethamine, proguanil (chloroguanide), spiramycin, sulfadoxine, artemisinin, dihydroartemisinin, artemether, artesunate, quinine derived from the bark of the South American chinchona tree, including quinine and quinine-related quinolines, halofantrine, mefloquine, lumefantrine, amodiaquine, pyronaridine, piperaquine, chloroquine, hydryoxychloroquine, napthoquine, primaquine, tafenoquine, 6-gingerol and 6-paradol, coronaridine and 18-methoxycoronaridine.

In certain embodiments the infection to be treated is caused by a protozoan parasite of the genera Giardia, Trichomonas, Leishmania, Trypanosoma, Crithidia, Herpetomonas, Leptomonas, Histomonas, Eimeria, Isopora, Neospora, or Plasmodium.

In certain embodiments the infection to be treated is malaria.

In certain embodiments the amount of naltrexone in the immediate release or sustained release formulation is between about 1.0 mg and about 8.0, preferably between about 0.05 mg and about 6.0 mg, more preferably between about 0.05 mg and about 4.5 mg.

In certain embodiments the administration of a sustained or immediate release formulation of naltrexone is once in a 24 hour period.

In certain embodiments wherein the administration of a sustained release formulation of naltrexone is once in a 24 hour period for about 7 days or about 30 days or about 90 days.

In certain embodiments the immediate release formulation releases the pharmaceutically acceptable salt of naltrexone completely within about 60 minutes.

In certain embodiments the route of administration is chosen from the group consisting of oral, sublingual, subcutaneous, intramuscular, intravenous, topical, local, intratracheal, intranasal, transdermal and rectal administration, preferably a capsule or tablet.

### Terms and Definitions Used

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include salts from ammonia, L-arginine, betaine, benethamine, benzathine, calcium hydroxide, choline, deanol, diethanolamine (2,2′-iminobis(ethanol)), diethylamine, 2-(diethylamino)-ethanol, 2-aminoethanol, ethylenediamine, N-ethyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, zinc hydroxide, acetic acid, 2,2-dichloro-acetic acid, adipic acid, alginic acid, ascorbic acid, L-aspartic acid, benzenesulfonic acid, benzoic acid, 2,5-dihydroxybenzoic acid, 4-acetamido-benzoic acid, (+)-camphoric acid, (+)-camphor-10-sulfonic acid, carbonic acid, cinnamic acid, citric acid, cyclamic acid, decanoic acid, dodecylsulfuric acid, ethane-1,2-disulfonic acid, ethanesulfonic acid, 2-hydroxy-ethanesulfonic acid, ethylenediaminetetraacetic acid, formic acid, fumaric acid, galactaric acid, gentisic acid, D-glucoheptonic acid, D-gluconic acid, D-glucuronic acid, glutamic acid, glutaric acid, 2-oxo-glutaric acid, glycerophosphoric acid, glycine, glycolic acid, hexanoic acid, hippuric acid, hydrobromic acid, hydrochloric acid, isobutyric acid, DL-lactic acid, lactobionic acid, lauric acid, lysine, maleic acid, (−)-L-malic acid, malonic acid, DL-mandelic acid, methanesulfonic acid, galactaric acid, naphthalene-1,5-disulfonic acid, naphthalene-2-sulfonic acid, 1-hydroxy-2-naphthoic acid, nicotinic acid, nitric acid, octanoic acid, oleic acid, orotic acid, oxalic acid, palmitic acid, pamoic acid (embonic acid), phosphoric acid, propionic acid, (−)-L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid. Further pharmaceutically acceptable salts can be formed with cations from metals such as aluminium, calcium, lithium, magnesium, potassium, sodium, zinc and the like (see Pharmaceutical salts, Berge, S. M. et al., J. Pharm. Sci., (1977), Vol. 66, pp. 1-19).

Pharmaceutically acceptable salts of the present invention can be synthesized from the parent compound, which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the naltrexone (e.g. trifluoro acetate salts), also comprise a part of the invention.

Typically, a pharmaceutically acceptable salt of a compound of naltrexone may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of naltrexone and the resulting mixture evaporated to dryness (lyophilized) to obtain the acid addition salt as a solid. Alternatively, a compound of naltrexone may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the compounds of naltrexone may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the invention.

Also included are both total and partial salts, that is to say salts with 1, 2 or 3, preferably 2, equivalents of base per mole of acid of formula I or salts with 1, 2 or 3 equivalents, preferably 1 equivalent, of acid per mole of base of formula I.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of naltrexone may form solvates (e.g., hydrates) and the invention also includes all such solvates. The meaning of the word “solvates” is well known to those skilled in the art as a compound formed by interaction of a solvent and a solute (i.e., solvation). Techniques for the preparation of solvates are well established in the art (see, for example, Brittain. Polymorphism in Pharmaceutical Solids. Marcel Decker, New York, 1999.).

The invention also encompasses prodrugs of the compounds of formula I, i.e., compounds which release an active parent drug (naltrexone) in vivo when administered to a mammalian subject. A prodrug is a pharmacologically active or more typically an inactive compound that is converted into a pharmacologically active agent by a metabolic transformation. Prodrugs of naltrexone are prepared by modifying functional groups present in naltraxone in such a way that the modifications may be cleaved in vivo to release the parent compound. In vivo, a prodrug readily undergoes chemical changes under physiological conditions (e.g., are acted on by naturally occurring enzyme(s)) resulting in liberation of the pharmacologically active agent. Prodrugs of naltraxone wherein a hydroxyl or amino, of naltrexone is bonded to any group that may be cleaved in vivo to regenerate the free hydroxyl, amino or carboxy group, respectively. Examples of prodrugs include esters (e.g., acetate, formate, and benzoate derivatives) of compounds of formula I or any other derivative, which upon being brought to the physiological pH or through enzyme action is converted to the active parent drug. Conventional procedures for the selection and preparation of suitable prodrug derivatives are described in the art (see, for example, Bundgaard. Design of Prodrugs. Elsevier, 1985).

Prodrugs may be administered in the same manner as the active ingredient to which they convert or they may be delivered in a reservoir form, e.g., a transdermal patch or other reservoir which is adapted to permit (by provision of an enzyme or other appropriate reagent) conversion of a prodrug to the active ingredient slowly over time, and delivery of the active ingredient to the patient.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

Naltrexone may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

### Pharmaceutical Compositions Comprising Naltraxone

While it is possible that naltrexone may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

Accordingly, the invention further provides a pharmaceutical composition comprising naltrexone or pharmaceutically acceptable salt thereof in admixture with a pharmaceutically acceptable carrier. The term “carrier” refers to a diluent, excipient, and/or vehicle with which an active compound is administered.

Naltexone may be used in combination with other therapies and/or active agents. Accordingly, the invention provides, in a further aspect, a pharmaceutical composition comprising naltrexone or a solvate, hydrate, enantiomer, diastereomer, N-oxide or pharmaceutically acceptable salt thereof, a second active agent, and a pharmaceutically acceptable carrier.

The pharmaceutical compositions may comprise as, in addition to, the carrier any suitable binder, lubricant, suspending agent, coating agent and/or solubilizing agent.

Preservatives, stabilizers, dyes and flavoring agents also may be provided in the pharmaceutical composition. Antioxidants and suspending agents may be also used.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see WO02/00196.

The term “immediate release” is defined as a release of compound from a dosage form in a relatively brief period of time, generally up to about 60 minutes.

### Routes of Administration and Unit Dosage Forms

The routes for administration include oral (e.g., as a tablet, capsule, or as an ingestible solution), topical, mucosal (e.g., as a nasal spray or aerosol for inhalation), nasal, parenteral (e.g., by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. The compositions of the invention may be especially formulated for any of those administration routes. In preferred embodiments, the pharmaceutical compositions of the invention are formulated in a form that is suitable for oral delivery.

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, the compound of Formula I may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

When appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the compositions may be administered in the form of tablets or lozenges, which can be formulated in a conventional manner.

When the composition of the invention is to be administered parenterally, such administration includes one or more of: intravenously, intra-arterially, intraperitoneally, intrathecally, intraventricularly, intraurethrally, intrasternally, intracranially, intramuscularly or subcutaneously administering the agent; and/or by using infusion techniques.

Pharmaceutical compositions of the invention can be administered parenterally, e.g., by infusion or injection. Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or acsorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound is best used in the form of a sterile aqueous solution, which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound of formula I with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the aldosterone receptor antagonists and desired excipients for subsequent preparation of sterile solutions. The compounds according to the invention may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilizing, solubilizing and/or dispersing agents. Alternatively, the active ingredient may be in sterile powder form for reconstitution with a suitable vehicle, e.g., sterile, pyrogen-free water, before use.

Naltrexone can be administered (e.g., orally or topically) in the form of tablets, capsules, ovules, elixirs, solutions or suspensions, which may contain flavoring or coloring agents, for immediate-, delayed-, modified-, sustained-, pulsed- or controlled-release applications.

Naltrexone may also be presented for human or veterinary use in a form suitable for oral or buccal administration, for example in the form of solutions, gels, syrups, mouth washes or suspensions, or a dry powder for constitution with water or other suitable vehicle before use, optionally with flavoring and coloring agents. Solid compositions such as tablets, capsules, lozenges, pastilles, pills, boluses, powder, pastes, granules, bullets or premix preparations may also be used. Solid and liquid compositions for oral use may be prepared according to methods well-known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The compositions may be administered orally, in the form of rapid or controlled release tablets, microparticles, mini tablets, capsules, sachets, and oral solutions or suspensions, or powders for the preparation thereof. In addition to the new solid-state forms of pantoprazole of the invention as the active substance, oral preparations may optionally include various standard pharmaceutical carriers and excipients, such as binders, fillers, buffers, lubricants, glidants, dyes, disintegrants, odourants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions include starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral compositions include acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable lubricants useful in the compositions of the invention include magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odourants for the oral compositions include synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include hydroxypropylmethylcellulose, hydroxypropylcellulose and acrylate-methacrylate copolymers.

Examples of pharmaceutically acceptable sweeteners for the oral compositions include aspartame, saccharin, saccharin sodium, sodium cyclamate, xylitol, mannitol, sorbitol, lactose and sucrose.

Examples of pharmaceutically acceptable buffers include citric acid, sodium citrate, sodium bicarbonate, dibasic sodium phosphate, magnesium oxide, calcium carbonate and magnesium hydroxide.

Examples of pharmaceutically acceptable surfactants include sodium lauryl sulphate and polysorbates. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Naltrexone may also, for example, be formulated as suppositories e.g., containing conventional suppository bases for use in human or veterinary medicine or as pessaries e.g., containing conventional pessary bases.

Naltrexone may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the agent of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colourants, and odourants.

Examples of pharmaceutically acceptable polymers suitable for such topical compositions include acrylic polymers; cellulose derivatives, such as carboxymethylcellulose sodium, methylcellulose or hydroxypropylcellulose; natural polymers, such as alginates, tragacanth, pectin, xanthan and cytosan. Examples of suitable pharmaceutically acceptable oils which are so useful include mineral oils, silicone oils, fatty acids, alcohols, and glycols.

Examples of suitable pharmaceutically acceptable liquid carriers include water, alcohols or glycols such as ethanol, isopropanol, propylene glycol, hexylene glycol, glycerol and polyethylene glycol, or mixtures thereof in which the pseudopolymorph is dissolved or dispersed, optionally with the addition of non-toxic anionic, cationic or non-ionic surfactants, and inorganic or organic buffers.

Examples of pharmaceutically acceptable preservatives include sodium benzoate, ascorbic acid, esters of p-hydroxybenzoic acid and various antibacterial and antifungal agents such as solvents, for example ethanol, propylene glycol, benzyl alcohol, chlorobutanol, quaternary ammonium salts, and parabens (such as methyl paraben, ethyl paraben and propyl paraben).

Examples of pharmaceutically acceptable stabilizers and antioxidants include ethylenediaminetetraacetic acid (EDTA), thiourea, tocopherol and butyl hydroxyanisole.

Examples of pharmaceutically acceptable moisturizers include glycerine, sorbitol, urea and polyethylene glycol.

Examples of pharmaceutically acceptable emollients include mineral oils, isopropyl myristate, and isopropyl palmitate.

The compounds may also be dermally or transdermally administered, for example, by use of a skin patch.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride.

As indicated, naltrexone can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

Naltrexone can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage form of the invention may be administered according to a dosage and administration regimen defined by routine testing in the light of the guidelines given above in order to obtain optimal activity while minimizing toxicity or side effects for a particular patient. However, such fine tuning of the therapeutic regimen is routine in the light of the guidelines given herein.

The dosage of the active agents of the invention may vary according to a variety of factors such as underlying disease conditions, the individual's condition, weight, gender and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician.

The pharmaceutical composition or unit dosage form may be administered in a single daily dose, or the total daily dosage may be administered in divided doses. In addition, co-administration or sequential administration of another compound for the treatment of the disorder may be desirable. To this purpose, the combined active principles are formulated into a simple dosage unit.

For combination treatment where the compounds are in separate dosage formulations, the compounds can be administered concurrently, or each can be administered at staggered intervals. For example, the compound of the invention may be administered in the morning and the antimuscarinic compound may be administered in the evening, or vice versa. Additional compounds may be administered at specific intervals too. The order of administration will depend upon a variety of factors including age, weight, gender and medical condition of the patient; the severity and aetiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

### DETAILED DESCRIPTION OF THE INVENTION

In accordance with the present invention, a n immediate or sustained release naltrexone composition preferably comprising between about 0.01 mg and about 10 mg of naltrexone may be administered to patients suffering protozoal infection or administered prophylactically such as to treat or prevent malaria in the form of an immediate release naltrexone tablets, liquids and cream, comprising from about 0.01 mg to about 10 mg of naltrexone in adults and about 0.05 to about 4.5 mg of naltrexone in children with suitable pharmaceutically-acceptable excipients, binders, sweeteners, coloring agents and other conventional additives.

The following examples provide a detailed illustration of the method of the present invention. These examples are not intended to limit or restrict the scope of the invention in any way.

### EXAMPLES

### Example 1 Prevention of Malaria by Naltrexone

45 BABL/C mice, six weeks old, were divided into 5 groups, 9 mice for each group. The groups were: normal saline group (negative group), infected group without treatment, 0.5 mg naltrexone group per mouse, 1 mg naltrexone group per mouse and 2 mg naltrexone group per mouse. Naltrexone was administrated orally everyday for successive 7 days.
1.5 h after administration on 7 d, the animals were infected with 106Py13XL malaria (P. falciparum). The survival was recorded from the first day of infection and infected rate of malaria are recorded from the third day of infection by taking tail vein blood, smearing glass slide, Gimsa staining and checking parasitemia under microscope.

### Results

In normal saline group (negative group), all animals are alive on 20 day.
In infected group without treatment, on 5 d 3 mice died, on 6 d, 5 more mice died and on 9 d, 1 more mouse dies (all mice died).
In 0.5 mg naltrexone group per mouse, on 5 d, 1 mouse died, on 6 d, 7 more mice died. 1 mouse is still alive at the end point.
In 1 mg naltrexone group per mouse, on 5 d, 1 mouse, on 6 d, 5 more mice died, 3 mice are still alive till the end point and on 20 d all infection parasitemia disappeared. The survival rate was 33.3%.
In 2 mg naltrexone group per mouse, on 5 d 4 mice died, on 6 d 5 more died.
From the results we can see that 1 mg naltrexone group per mouse shows the best protecting effect and finally all infected animals left return to normal.

### Example 2 Anti-Malaria Effect with Naltrexone

45 BABL/C mice, six weeks old, were divided into 5 groups, 9 mice for each group. They are: normal saline group (negative group), infected group without treatment, 0.5 mg naltrexone treating group, 1 mg naltrexone treating group and 2 mg naltrexone treating group.
The animals were infected with 105Py13XL malaria. On 2 d post infection, naltrexone was administrated orally everyday for successive 15 days. The survival was recorded from the third day of infection and infected rate of malaria are recorded by taking tail vein blood, smearing glass slide, Gimsa staining and checking parasitemia under microscope.
Finally changes of immune parameters in peripheral blood were measured by flow cytometry technology and made comparison to control.

### Results

In normal saline group (negative group), all animals are alive on 20 day.
In infected group without treatment, on 5 d 4 mice died, on 6 d 4 more mice died and on 9 d 1 more mouse dies (all mice died).
In 0.5 mg naltrexone treating group, on 5 d 2 mouse died, on 6 d 4 more mice died. 3 mouse is still alive for end point.
In 1 mg naltrexone treating group, on 5 d 0 mouse, on 6 d 2 more mice died, on 9 d 1 more mouse died. 6 mice are still alive till end point and on 20 d all infection, parasitemia disappeared.
In 2 mg naltrexone group, on 5 d 5 mouse died, on 6 d 4 more died.
From the results we can see that 1 mg naltrexone group per mouse shows the best treatment. 2 mg shows toxicity to animals.
After treatment with low dose naltrexone

### Changes of Immune Parameters in Peripheral Blood Post Treat

| | Control | Treatment | P |
|---|---|---|---|
| CD3+ T cell | 48.90 ± 4.06 | 67.04 ± 4.20 | P < 0.01 |
| CD8+ T cell | 35.33 ± 3.62 | 59.63 ± 3.78 | P < 0.01 |
| CD4+ T cell | 22.65 ± 3.48 | 34.81 ± 5.78 | P < 0.01 |
| NK cell | 7.58 ± 1.59 | 20.39 ± 4.42 | P < 0.01 |
| Treg cell | 4.22 ± 0.64 | 2.15 ± 0.48 | P < 0.01 |
| Dnedritic cell | 7.22 ± 0.34 | 11.7 ± 0.32 | P < 0.01 |
| Macrophage | 9.18 ± 0.26 | 12.1 ± 0.56 | P < 0.01 |
| Total nucleated cell | 1.30 ± 0.13 | 4.21 ± 0.75 | P < 0.01 |

### Example 3

A 57 year old male patient with HIV suffering from malaria has had relapses at least twice a year for the last five years. The patient was administered LDN (low dose naltrexone) at 4.5 mg per day as an adjunct treatment for HIV. The patient has not had a relapse of malaria in 24 months.

### Example 4

A 65 year old woman has suffered on and off from malaria for 40 years. The patient was administered 4.5 mg of LDN per day, as an adjunct treatment with chemotherapy. The patient has not had a relapse of malaria for over 24 months.
References cited are herein incorporated by reference in their entireties.

1. A method for treating or preventing protozoan parasite infection comprising administering to a mammal in need thereof naltrexone or a pharmaceutically acceptable salt thereof alone or in combination with one or more anti-protozoal agents.

2. The method of claim 1 wherein the anti-protozoal agent is chosen from the group consisting of atovaquone, amodiaquine, amphotericin, butoconazole, clindamycin, eflornithine, fumagillin, iodoquinol (diiodohydroxyquin), clioquinol (iodochlorhydroxyquin), Etanidazole, Benznidazole, fluoroquinolones, enoxacin, ciprofloxacin, doxycycline, tetracycline, melarsoprol, metronidazole, miltefosine, nifurtimox, nitazoxanide, paromomycin, pentamindine, sodium stibogluconate, suramin, tinidozole, pyrimethamine, proguanil (chloroguanide), spiramycin, sulfadoxine, artemisinin, dihydroartemisinin, artemether, artesunate, quinine derived from the bark of the South American chinchona tree, including quinine and quinine-related quinolines, halofantrine, mefloquine, lumefantrine, amodiaquine, pyronaridine, piperaquine, chloroquine, hydryoxychloroquine, napthoquine, primaquine, tafenoquine, 6-gingerol and 6-paradol, coronaridine and 18-methoxycoronaridine.

3. The method of claim 1 wherein the infection is caused by a protozoan parasite of the genera Giardia, Trichomonas, Leishmania, Trypanosoma, Crithidia, Herpetomonas, Leptomonas, Histomonas, Eimeria, Isopora, Neospora, or Plasmodium.

4. The method of claim 1 wherein the infection is malaria.

5. A method for treating or preventing protozoan parasite infection comprising administering to a mammal in need thereof an immediate release pharmaceutical formulation comprising naltrexone or a pharmaceutically acceptable salt thereof alone or in combination with one or more anti-protozoal agents.

6. The method of claim 5 wherein the anti-protozoal agent is chosen from the group consisting of atovaquone, amodiaquine, amphotericin, butoconazole, clindamycin, eflornithine, fumagillin, iodoquinol (diiodohydroxyquin), clioquinol (iodochlorhydroxyquin), Etanidazole, Benznidazole, fluoroquinolones, enoxacin, ciprofloxacin, doxycycline, tetracycline, melarsoprol, metronidazole, miltefosine, nifurtimox, nitazoxanide, paromomycin, pentamindine, sodium stibogluconate, suramin, tinidozole, pyrimethamine, proguanil (chloroguanide), spiramycin, sulfadoxine, artemisinin, dihydroartemisinin, artemether, artesunate, quinine derived from the bark of the South American chinchona tree, including quinine and quinine-related quinolines, halofantrine, mefloquine, lumefantrine, amodiaquine, pyronaridine, piperaquine, chloroquine, hydryoxychloroquine, napthoquine, primaquine, tafenoquine, 6-gingerol and 6-paradol, coronaridine and 18-methoxycoronaridine.

7. The method of claim 5 wherein the infection is caused by a protozoan parasite of the genera Giardia, Trichomonas, Leishmania, Trypanosoma, Crithidia, Herpetomonas, Leptomonas, Histomonas, Eimeria, Isopora, Neospora, or Plasmodium.

8. The method of claim 5 wherein the infection is malaria.

9. The method of claim 5 wherein the amount of naltrexone is between about 0.01 mg and about 10.0 mg.

10. The method of claim 5 wherein the amount of naltrexone is between about 1.0 mg and about 8.0 mg.

11. The method of claim 5 wherein the amount of naltrexone is between about 0.05 mg and about 6.0 mg.

12. The method of claim 5 wherein the amount of naltrexone is between about 0.05 mg and about 4.5 mg.

13. The method of claim 5 wherein said administration is once in a 24 hour period.

14. The method of claim 5 wherein said administration is once in a 24 hour period for between about 7 to about 90 days.

15. The method according to claim 14 wherein said administration is once in a 24 hour period for about 7 days.

16. The method according to claim 14 wherein said administration is once in a 24 hour period for about 30 days.

17. The method according to claim 14 wherein said administration is once in a 24 hour period for about 90 days.

18. The method of claim 5 wherein said mammal is a human.

19. The method of claim 5 wherein said pharmaceutically acceptable salt is the hydrochloride salt.

20. The method of claim 5 wherein said immediate release composition releases the pharmaceutically acceptable salt of naltrexone completely within about 60 minutes.

21. The method of claim 5 wherein said administration is chosen from the group consisting of oral, sublingual, subcutaneous, intramuscular, intravenous, topical, local, intratracheal, intranasal, transdermal and rectal administration.

22. The method of claim 5 wherein said immediate release composition is in the form of a capsules or tablet.

23-40. (canceled)
